(19) United States
(12) Patent Application Publication
(10) Pub. No.: US 2010/0331652 A1
(43) Pub. Date: Dec. 30, 2010

(54) MODULAR DIABETES MANAGEMENT SYSTEMS

(75) Inventors: Henning Groll, Oro Valley, IN (US); Nigel Surridge, Indianapolis, IN (US)

Correspondence Address: KRIEG DEVAULT/ROCHE, 2800 ONE INDIANA SQUARE, INDIANAPOLIS, IN, 46204, US

(73) Assignee: Roche Diagnostics Operations, Inc., Indianapolis, IN (US)

(21) Appl. No.: 12/493,545

(22) Filed: Jun. 29, 2009

(51) Int. Cl. A61B 5/145

(52) U.S. Cl. 600/365

(57) ABSTRACT

In one embodiment, a modular diabetes management system includes a portable, stand-alone blood glucose meter and a portable docking device that includes an internal receptacle sized and structured to receive and house the blood glucose meter. The docking device is generally operable to interface with the blood glucose meter and perform various diabetes management functions. For example, the docking device may be operable to analyze blood glucose measurement data stored on the blood glucose meter, configure the blood glucose meter, and/or interact with an insulin delivery device, just to name a few possibilities. Other embodiments include unique methods, systems, kits, assemblies, equipment, and/or apparatus which are related to the management of diabetes.

### BACKGROUND

As the number of patients suffering from diabetes and similar medical conditions increases, self-monitoring of blood glucose wherein the patient monitors his or her blood glucose levels has become a common practice. The purpose of monitoring the blood glucose level is to determine the concentration level and then to take corrective action, based upon whether the level is too high or too low, to bring the level back within a normal range. The failure to take corrective action can have serious medical implications. Glucose monitoring is a fact of everyday life for diabetic individuals. Failure to test blood glucose levels properly and on a regular basis can result in serious diabetes-related complications, including cardiovascular disease, kidney disease, nerve damage and blindness.

People with diabetes who intensively manage their blood sugar experience long-lasting benefits. The Diabetes Control and Complications Trial (DCCT) was a clinical study conducted from 1983 to 1993 by the National Institute of Diabetes and Digestive and Kidney Diseases (NIDDK). The DCCT compared intensive to conventional treatments. Patients on intensive treatment kept glucose levels as close to normal as possible with at least three insulin injections a day or an insulin pump, and frequent self-monitoring of blood glucose. Intensive treatment aimed to keep hemoglobin A1c (HbA1c), which reflects average blood glucose over a 2- to 3-month period, as close to normal as possible. Intensive treatment aimed to keep hemoglobin A1c (HbA1c), which reflects average blood glucose over a 2- to 3-month period, as close to normal as possible. Conventional treatment consisted of one or two insulin injections a day with once-a-day urine or blood glucose testing. The results of the DCCT study showed that keeping blood glucose levels as close to normal as possible slows the onset and progression of eye, kidney, and nerve diseases caused by diabetes. In fact, it demonstrated that any sustained lowering of blood glucose helps, even if the person has a history of poor control.

A number of glucose meters are currently available that permit an individual to test the glucose level in a small sample of blood. Many of the meter designs currently available make use of a disposable test element which in combination with the meter measures the amount of glucose in the blood sample electrochemically or optically. In current glucose meters, the information displayed as a consequence of a successful blood glucose measurement is the respective blood glucose value, typically shown in mg/dL or mmol units, and perhaps the time and date the measurement was performed. This information in combination with calculation of planned or known intake of carbohydrates or planned or known activities and knowledge of other situational or individual factors is in most cases sufficient to allow diabetics to adjust or derive their dietary intake and/or an immediate dose of insulin to inject to control blood glucose level on the short-term. Also, in case of low glucose values, diabetics can detect the need for intake of sugar to avoid hypoglycemia. Further, analysis of multiple blood glucose measurements taken over a period of time assists diabetes patients in determining what, if any, long term changes are necessary for their diabetes management routine. Given the ramifications of accurate recording, reporting and analyzing of blood glucose measurements, improvements in the apparatus and/or procedures to meter blood glucose are desired.

### SUMMARY

A system includes a stand-alone blood glucose meter that is positionable within an internal receptacle of a portable, handheld docking device. The docking device is configured to interface with the blood glucose meter to facilitate user configuration of the blood glucose meter and analysis of blood glucose measurement data stored on the blood glucose meter. Among other things, the interface between the docking device and the blood glucose meter reduces the time and complexity currently associated with blood glucose management.

In one aspect, a system includes a portable, stand-alone blood glucose meter including a display and a first connection element. The system also includes a portable, handheld docking device including a display operable to provide information related to functions performed by said blood glucose meter and a housing defining an external profile of the docking device and an internal receptacle. The internal receptacle includes a second connection element engageable with the first connection element. The blood glucose meter is positionable in the internal receptacle and a communication interface is defined between the blood glucose meter and the docking device when the blood glucose meter is positioned in the internal receptacle and the first connection element is engaged with the second connection element.

In one refinement of the aspect the blood glucose meter includes a meter housing which substantially corresponds in size and shape to the internal receptacle. When the blood glucose meter is positioned in the internal receptacle the meter housing is substantially surrounded by the internal receptacle and located within the external profile of the docking device.

In another refinement of the aspect the housing of the docking device includes a sidewall extending between a first surface and a second surface positioned opposite of the first surface and the internal receptacle is positioned between the first and second surfaces.

In another refinement of the aspect the internal receptacle includes a receiving portion extending through the sidewall of the housing.

In another refinement of the aspect the second surface includes an opening in communication with a portion of the internal receptacle adjacent to the receiving portion.

In another refinement of the aspect the second connection element is positioned in the internal receptacle opposite of the receiving portion.

In another refinement of the aspect one of the first connection element and the second connection element defines a plug and the other of the first connection element and the second connection element defines a port structured to receive the plug.

In another refinement of the aspect the blood glucose meter includes a meter housing defining a test element port for receiving a test element. An appropriate test element may comprise the form of a test strip configured for either electrochemical or optical techniques of detection of blood glucose concentration.

In another refinement of the aspect the first connection element is positioned on the meter housing opposite of the test element port.

In another refinement of the aspect the blood glucose meter includes a memory configured to store blood glucose measurement data.

In another refinement of the aspect the blood glucose measurement data is transferred from the blood glucose meter to the docking device when the blood glucose meter is positioned in the internal receptacle and the first connection element is engaged with the second connection element. Such a data transfer may occur generally automatically, e.g. seamlessly.

In another refinement of the aspect the docking device is configured to process the blood glucose measurement data and provide an output to a user of the docking device after the blood glucose measurement is processed.

In another refinement of the aspect the output is a graphical representation of the blood glucose measurement data.

In another refinement, the stand-alone blood glucose meter is configured to perform blood glucose measurements on a blood sample supplied to a test element, whether used as a stand-alone device or while positioned in the internal receptacle of the docking device with the first and second connections engaged. In the case of the latter, currently determined blood glucose measurement data is displayed directly on the display of the docking device.

In another refinement, a blood glucose measurement is provided on the display of the blood glucose meter in response to measuring a glucose level with the blood glucose meter when the glucose meter is not positioned in the internal receptacle of the docking device.

In another refinement, a blood glucose measurement is provided on the display of the docking device in response to measuring a glucose level with the blood glucose meter when the blood glucose meter is positioned in the internal receptacle of the docking device.

In a further aspect, a system includes a portable, stand alone blood glucose meter and a plurality of portable, handheld docking devices. Each of the docking devices includes a housing defining an internal receptacle sized and structured to receive the blood glucose meter. The internal receptacle also includes an interface for communicating with the blood glucose meter when the blood glucose meter is positioned therein. Each one of the plurality of docking devices is operable to communicate with the blood glucose meter to perform a unique set of diabetes management functions relative to the other of the plurality of docking devices.

In one refinement of the aspect the blood glucose meter and each one of the plurality of docking devices further includes a display, a user entry means for receiving user input, a memory and a programmable processor operatively connected to the display, the user entry means and the memory.

In another refinement of the aspect the memory of the blood glucose meter stores a program for operating the processor of the blood glucose meter. The processor of the blood glucose meter is operable with the program to produce at least one blood glucose measurement, provide the at least one blood glucose measurement on the display of the blood glucose meter and store the at least one blood glucose measurement in the memory of the blood glucose meter.

In another refinement of the aspect the memory of the blood glucose meter includes a calendar that stores a schedule of events and the processor of the blood glucose meter is operable with the program to activate a reminder of each of the events to a user.

In another refinement of the aspect at least one of the plurality of docking devices is operable to organize the schedule of events when the blood glucose meter is positioned in the internal receptacle of the at least one docking device.

In another refinement of the aspect the schedule of events includes a plurality of predetermined times for measuring blood glucose levels.

In another refinement of the aspect the user entry means of the blood glucose meter includes a plurality of buttons.

In another refinement of the aspect the processor of the blood glucose meter is operable with the program upon activation of a first one of the plurality of buttons by a user to activate a reminder for measuring blood glucose levels after one of a plurality of predefined periods of time.

In another refinement of the aspect the one of a plurality of predefined periods of time is determined by a number of times a user activates the first one of the plurality of buttons.

In another refinement of the aspect the memory of each of the docking devices stores a unique management program for operating the processor of the respective docking device.

In another refinement of the aspect the processor of at least one of the plurality of docking devices is operable to process the at least one blood glucose measurement stored in the memory of the blood glucose meter.

In another refinement of the aspect the processor of the at least one of the plurality of docking devices is further operable to perform one or more of the disease management functions after the at least one blood glucose measurement is processed.

In another refinement of the aspect the disease management functions include at least one of providing a graphical representation of the at least one blood glucose measurement and providing instructions to an insulin delivery device.

In another refinement of the aspect the processor of the at least one of the plurality of docking devices provides a graphical representation of the at least one blood glucose measurement when the blood glucose meter is positioned in the internal receptacle of the at least one of the plurality of docking devices. In one form of this refinement, the graphical representation may generally be provided automatically, e.g. seamlessly.

In another refinement of the aspect the processor of at least one of the plurality of docking devices is operable with a respective management program to format the program for operating the processor of the blood glucose meter.

In another refinement of the aspect the user entry means of at least one of the plurality of docking devices is defined by one or more of a touch screen, a camera and a voice recorder.

In another aspect, a method includes providing a portable, handheld docking device including a display and a housing defining an external profile of the docking device and an internal receptacle structured to house a stand-alone blood glucose meter; positioning the blood glucose meter in the internal receptacle while substantially retaining the external profile of the docking device; and in response to the positioning, transferring information from at least one of the docking device and the blood glucose meter to the other of the docking device and the blood glucose meter.

In one refinement of the aspect the positioning includes engaging a first connection element of the blood glucose meter with a second connection element positioned in the internal receptacle.

In another refinement of the aspect the transferring includes receiving blood glucose measurement data from the blood glucose meter with the docking device. In one form of this refinement, the blood glucose measurement data is generally received automatically, e.g. seamlessly.

In another refinement the aspect further includes, in response to receiving the blood glucose measurement data, processing the blood glucose measurement data with the docking device.

In another refinement the aspect further includes, in response to processing the blood glucose measurement data, at least one of producing a graphical representation of the blood glucose measurement data on the display of the docking device and providing instructions with the docking device to an insulin delivery device.

In another refinement of the aspect the transferring includes loading one or more events with the docking device onto a schedule stored in the blood glucose meter.

In another refinement of the aspect providing the docking device includes selecting the docking device from a plurality of portable, handheld docking devices, each of the plurality of docking devices including a display and a housing defining an external profile of the respective docking device and an internal receptacle structured to house the blood glucose meter.

In another refinement of the aspect each of the plurality of docking devices is independently configured to provide a unique set of diabetes management functions relative to the other of the plurality of docking devices.

Another refinement of the aspect includes providing a blood glucose measurement on the display of the docking device in response to measuring a blood glucose level with the blood glucose meter when the blood glucose meter is positioned in the internal receptacle of the docking device.

Another refinement of the aspect includes removing the blood glucose meter from the internal receptacle, measuring a blood glucose level with the blood glucose meter and, in response to measuring the blood glucose level, providing a blood glucose measurement on a display of the blood glucose meter.

In another aspect, a system includes a portable, stand-alone blood glucose meter. The system also includes a portable, handheld docking device including a display and a housing defining an external profile of the docking device and an internal receptacle in which the blood glucose meter is positionable. When the blood glucose meter is positioned in the internal receptacle a communication interface is defined between the blood glucose meter and the docking device and the external profile of the docking remains substantially unchanged.

In another aspect, a system includes a portable, stand-alone blood glucose meter. The system also includes a portable, handheld docking device including a display and a housing. The housing includes a sidewall extending between upper and lower surfaces. An internal receptacle is defined by the housing between the upper and lower surfaces and is sized and configured to receive the blood glucose meter. When the blood glucose meter is positioned in the internal receptacle a communication interface is defined between the blood glucose meter and the docking device.

Another aspect of the present application is a unique system for testing and monitoring blood glucose levels of a diabetic. Other aspects include unique methods, systems, devices, kits, assemblies, equipment, and/or apparatus related to measuring blood glucose levels.

Further aspects, embodiments, forms, features, benefits, objects, and advantages shall become apparent from the detailed description and figures provided herewith.

### BRIEF DESCRIPTION OF THE FIGURES

FIG. 1 is a partially-exploded, perspective view of one embodiment of a modular diabetes management system including a blood glucose meter and a docking device.

FIG. 2 is a perspective view of the blood glucose meter of the system of FIG. 1 performing a blood glucose measurement.

FIG. 3 is a plan view of the blood glucose meter of the system of FIG. 1.

FIG. 4 is a schematic of the blood glucose meter of the system of FIG. 1.

FIG. 5 is a perspective view of the modular diabetes management system of FIG. 1 with the blood glucose meter engaged with the docking device.

FIG. 6 is an alternative perspective view of the modular diabetes management system of FIG. 1 with the blood glucose meter engaged with the docking device.

FIG. 7 is a schematic of the docking device of the system of FIG. 1.

FIG. 8 illustrates an example of a scheduling program on the display of the docking of FIG. 1.

FIG. 9 is a perspective view of the modular diabetes management system of FIG. 1 with the blood glucose meter engaged with the docking device and positioned adjacent to a meal.

FIG. 10 illustrates an example of a diary log on the display of the docking of FIG. 1.

FIG. 11 illustrates an example of a training video on the display of the docking device of FIG. 1.

FIG. 12 illustrates a system that includes a plurality of docking devices engageable with a blood glucose meter.

### DETAILED DESCRIPTION OF THE ILLUSTRATED EMBODIMENTS

For purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In order to control and more effectively adjust the therapy, level of activity and lifestyle to achieve optimum glycemic control, diabetics need to measure blood glucose levels on a regular basis. Moreover, information based on more than one blood sugar measurement can be useful to diabetics and their health care providers to establish trends, variability, running or absolute averages which can assist in adjusting behavior as well as medication for both insulin and non-insulin dependent diabetics. Various embodiments of the present invention are related to a modular diabetes management system that addresses these and other aspects. In one embodiment, the system includes a portable, stand-alone blood glucose meter and a portable docking device that includes an internal receptacle sized and structured to receive and house the blood glucose meter. The docking device is generally operable to interface with the blood glucose meter and perform various diabetes management functions. For example, the docking device may be operable to analyze blood glucose measurement data stored on the blood glucose meter, configure the blood glucose meter, and/or interact with an insulin delivery device, just to name a few possibilities. Still, further aspects and features of the present application are described with respect to the illustrated embodiments as follows.

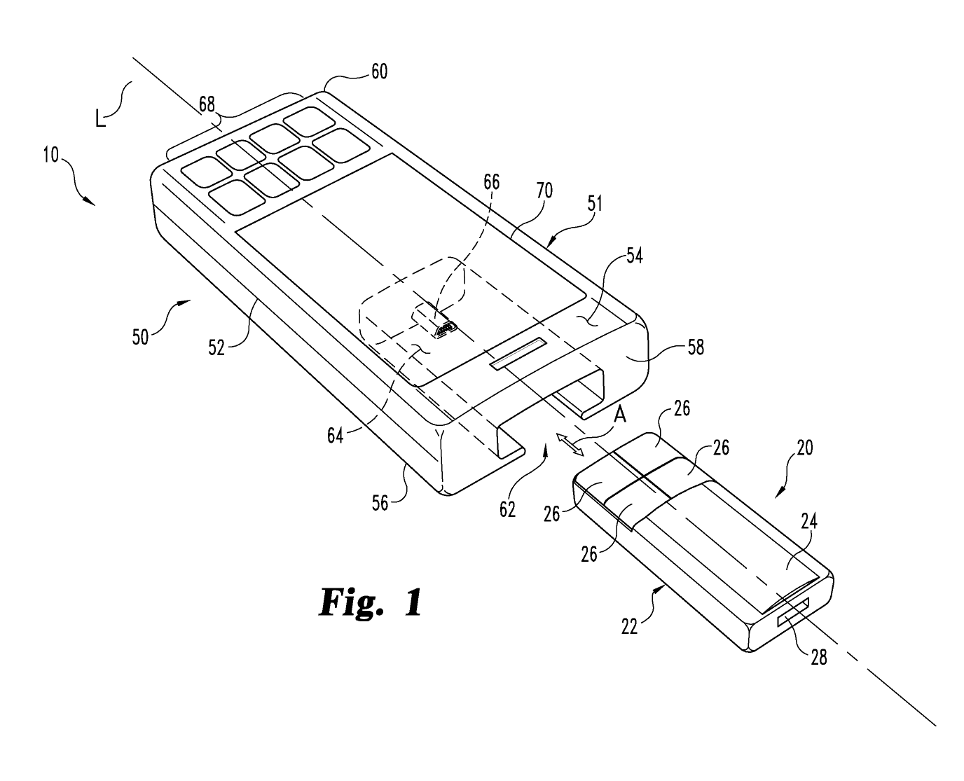

Referring to FIG. 1, there is illustrated a modular diabetes management system 10 that includes a portable, stand-alone blood glucose (bG) meter 20 and a portable docking device 50. The bG meter 20 is positionable in engagement with docking device 50 to provide a communication interface therebetween. Further details of docking device 50 and the interface between it and meter 20 will be provided below.

Referring now generally to FIGS. 1-3, bG meter 20 is operable for testing and measuring glucose levels in the blood of a user from a sample applied to a test element using electrochemical or optical techniques. An example of a test element configured for use with electrochemical techniques is the ACCU-CHEK® Aviva test strip, which is described more fully in U.S. Patent Application Publication No. 2005/0016844, the disclosure of which is hereby incorporated herein by reference in its entirety; and an example of a test element configured for use with optical techniques is the ACCU-CHEK® Compact test strip, which is described more fully in U.S. Pat. No. 7,008,799, the disclosure of which is hereby incorporated herein by reference in its entirety. Each of these exemplary test elements are distributed in the United States by Roche Diagnostics Corporation of Indianapolis, Ind.

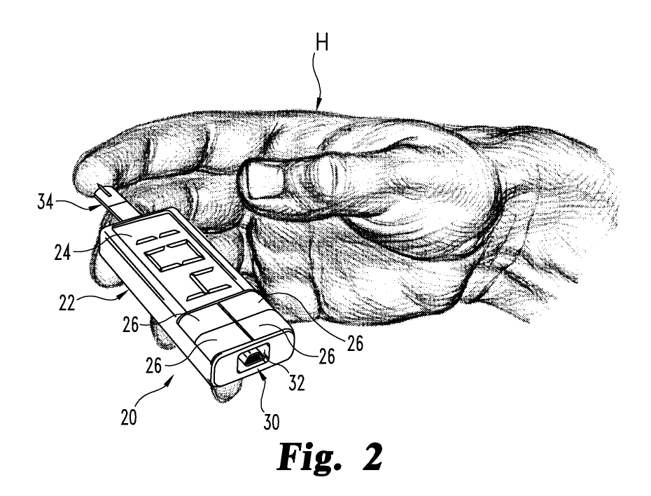

The bG meter 20 includes a housing 22 with a capable display 24, user entry means 26, and a test element port 28. It should be appreciated that housing 22 can be sufficiently compact so that it can be conveniently hand held and carried by the user. Electronic circuitry is contained within housing 22 to provide an electrochemical or optical measurement of a glucose level from a sample of blood on a test element inserted into test element port 28. For example, as illustrated in FIG. 2, an electrochemical test element in the form of test strip 34 has been inserted into test element port 28 and bG meter 20 has performed a glucose measurement of blood taken from a finger of hand H of the user. The results of the bG measurement performed by meter 20 are provided on display 24 in FIG. 2.

Further details and examples of conventional bG meters and related electrical and optical components and their respective measurement techniques are described in U.S. Pat. Nos. 5,352,351; 4,999,482; 5,438,271; 6,645,368; 5,997,817; 6,662,439; RE 36,268; 5,463,467; 5,424,035; 6,055,060; 6,906,802; and 5,889,585; the disclosures of which are hereby incorporated herein by reference in their entireties.

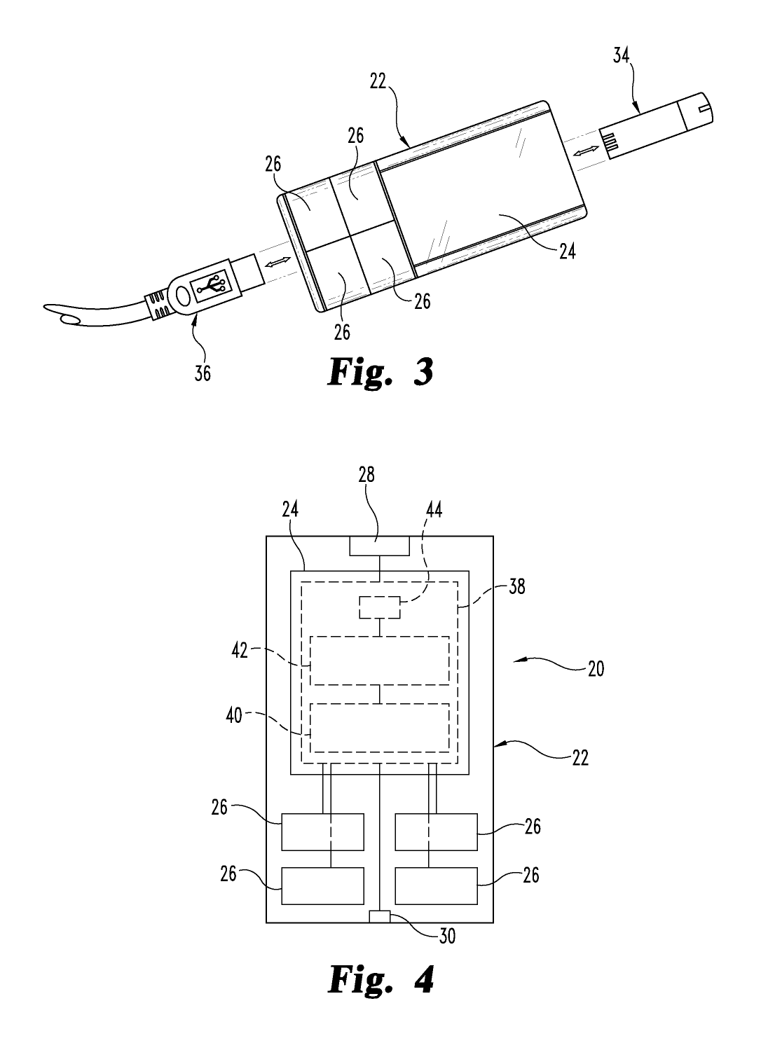

Housing 22 of bG meter 20 also includes a connection element 30 positioned opposite of test element port 28. Connection element 30 is generally structured to engage with a corresponding connection element of docking device 50, further details of which are provided below. In the illustrated embodiment of bG meter 20, connection element 30 is in the form of a mini-USB port 32 structured to receive a mini-USB plug, such as plug 36 illustrated in FIG. 3, although alternative configurations of connection element 30 are contemplated. It should be appreciated that plug 36 may be representative of the connection element of docking device 50 or some other communication link, such as a cord, which is utilized to interface bG meter 20 with a secondary device such as a PC. Additionally, in one or more non-illustrated embodiments, it is contemplated that connection element 30 may be in the form of a plug structured to engage with a corresponding port on docking device 50 or some other secondary device. The bG meter 20 may also include one or more other compartments or features for storage of lancets, test elements such as test strips 34, cleaning swabs or other items (not shown) which may be useful with bG meter 20.

In FIG. 4 there is shown one embodiment of a schematic of an exemplary bG meter 20 that is suitable for use with system 10. The bG meter 20 includes a controller 38, memory 40 associated with controller 38, a programmable processor 42 associated with controller 38 and connected with memory 40, and a real-time clock 44 associated with controller 38 and connected with processor 42. Display 24 is connected with processor 42 with, for example, a display driver, and operable to provide a user readable display of output from processor 42. User entry means 26 is connected with processor 42 and accessible by the user to provide input to processor 42. Processor 42 is connected with test element port 28 and operable to process and record data in memory 40 relating to a blood glucose measurement taken in test element port 28 and produce a representation of the current bG measurement and associated data on display 24. Processor 42 is further programmable to receive input commands from user entry means 26 and provide an output that responds to the input commands. Processor 42 is also connected with connection element 30 and is operable to control the transfer of information to and from bG meter 20.

Controller 38 may be comprised of one or more components configured as a single unit or of multi-component form. Controller 38 may be programmable, a state logic machine or other type of dedicated hardware, or a hybrid combination of programmable and dedicated hardware. One or more components of controller 38 may be of the electronic variety defining digital circuitry, analog circuitry, or both. As an addition or alternative to electronic circuitry, controller 38 may include one or more mechanical or optical control elements.

In one embodiment including electronic circuitry, controller 38 includes an integrated processor 42 operatively coupled to one or more solid-state memory devices defining, at least in part, memory 40. For this embodiment, memory 40 contains operating logic to be executed by a processor 42 that is a microprocessor and is arranged for reading and writing of data in memory 40 in accordance with one or more routines of a program executed by microprocessor 42.

Memory 40 may include one or more types of solid-state electronic memory and additionally or alternatively may include the magnetic or optical variety. For example, memory 40 may include solid-state electronic Random Access Memory (RAM), Sequentially Accessible Memory (SAM) (such as the First-In, First-Out (FIFO) variety or the Last-In First-Out (LIFO) variety), Programmable Read. Only Memory (PROM), Electrically Programmable Read Only Memory (EPROM), or Electrically Erasable Programmable Read Only Memory (EEPROM); or a combination of any of these types. Also, memory 40 may be volatile, nonvolatile or a hybrid combination of volatile and nonvolatile varieties. Some or all of memory 40 can be of a portable type, such as a disk, tape, memory stick, cartridge, code chip or the like. Memory 40 can be at least partially integrated with processor 42 and/or may be in the form of one or more components or units.

In other embodiments, it is contemplated that the bG meter 20 may utilize a removable memory key that is pluggable into a socket or other receiving means (not shown) of housing 22, and which communicates with the memory or controller of the meter 20 to provide information relating to calibration codes, measurement methods, measurement techniques, and information management. Examples of such removable memory keys are disclosed in U.S. Pat. Nos. 5,366,609 and 5,053,199, the disclosures of which are incorporated herein by reference in their entireties.

In one embodiment, memory 40 of bG meter 20 includes a calendar that stores a schedule of events which may be representative of times for measuring bG levels, taking medications, visiting a physician or performing other daily tasks, including attending work, school and/or meetings, just to name a few possibilities. In this embodiment, processor 42 is programmed so that an alarm or other indicator on bG meter 20 audibly or by vibration alerts the user to perform each event according to the schedule. Once the event has been completed, a user may provide an indication of same with user entry means 26 and processor 42 is programmed to process and record the indication in memory 40. In one form, one of user entry means 26 is designated a reminder input. Upon user interaction with the reminder input, processor 42 is programmed to initiate a countdown of a period of time until it activates an alarm or other indicator. Once the countdown is complete, processor 42 is programmed so that an alarm or other indicator on bG meter 20 audibly or by vibration alerts the user to perform the event. In one form, the event is measuring a bG level. In another form, the length of the countdown is determined by the number of times a user interacts with the reminder input within a predefined period of time. For example, a single occurrence of user interaction with the reminder input can set the countdown at one hour while a double occurrence of user interaction with the reminder input can set the countdown at two hours. However, it should be appreciated that different lengths of the countdown as well as arrangements for determining the length of the countdown are contemplated.

Besides memory 40, controller 38 may also include clock 44, display 24, and entry means 26 associated therewith, along with signal conditioners, filters, limiters, Analog-to-Digital (A/D) converters, Digital-to-Analog (D/A) converters, communication ports, or other types of operators as would occur to those skilled in the art to implement the present invention. In the illustrated embodiment, entry means 26 is defined by a plurality of push-button input devices, although entry means 26 may include one or more other types of input devices like a keyboard, mouse or other pointing device, touch screen, touch pad, roller ball, or a voice recognition input subsystem. Display 24 may include one or more output means like an operator display that can be of a Cathode Ray Tube (CRT) type, Liquid Crystal Display (LCD) type, plasma type, Organic Light Emitting Diode (OLED) type, a printer, or the like. Other input and display means can be included such as loudspeakers, voice generators, voice and speech recognition systems, haptic displays, electronic wired or wireless communication subsystems, and the like.

As illustrated in FIG. 1 for example, docking device 50 includes a housing 51 which defines its external profile. It should be appreciated that housing 51 can be sufficiently compact so that it can be conveniently hand held and carried by the user. Housing 51 is defined by a sidewall 52 extending around docking device 50 along its elongated sides and first and second ends 58, 60 and between an upper surface 54 and a lower surface 56. In one embodiment, sidewall 52 is formed of a unitary construction, although other arrangements of sidewall 52 are contemplated. As illustrated, the external profile of docking device 50 has a substantially parallelepiped prism configuration. In this arrangement, first and second ends 58, 60 are similarly sized and shaped and surfaces 54 and 56 are also similarly sized and shaped. It should be appreciated however that other configurations for the external profile of docking device 50 are contemplated. Docking device 50 defines an internal receptacle 64 (shown in phantom) extending along a longitudinal axis L of docking device 50 from first end 58 of docking device 50 toward second end 60 of docking device 50. Internal receptacle 64 is positioned between upper surface 54 and lower surface 56 and generally corresponds in size and shape to the external profile of meter housing 22 of bG meter 20. Additionally, in the illustrated embodiment, internal receptacle 64 underlies and is positioned adjacent to at least a portion of display 70, although alternative configurations for the positioning internal receptacle relative to display 70 are contemplated. As indicated by directional arrow A, bG meter 20 is removably positionable within internal receptacle 64 of docking device 50. At first end 58 of docking device 50, internal receptacle 64 extends through sidewall 52 and defines a receiving portion 62 in the form of a window or opening. While not illustrated, it is contemplated that receiving portion 62 can be structured to initiate engagement of bG meter 20 with docking device 50 in a pre-determined alignment. For example, receiving portion 62 can include one or more tapered or angled surfaces configured to direct bG meter 20 into internal receptacle 64. Similarly, while not illustrated, it is contemplated that meter housing 22 of bG meter 20 can have corresponding tapered or angled surfaces to promote its engagement with internal receptacle 64 in the pre-determined alignment. Docking device 50 may also include one or more other compartments or features for storage of lancets, test elements such as test strips 34, cleaning swabs or other items (not shown) which may be useful with system 10.

A connection element 66 (shown in phantom) is positioned in internal receptacle 64 opposite of receiving portion 62. Connection element 66 is structured to physically engage with connection element 30 of bG meter 20. More particularly, as bG meter 20 is positioned into internal receptacle 64, connection element 66 makes contact with and gradually engages connection element 30 of bG meter 20 to define a communication interface between bG meter 20 and docking device 50 once bG meter 20 is fully positioned in internal receptacle 64. In one form, connection element 66 is a mini-USB plug similar to plug 36 to facilitate engagement with mini-USB port 32 on bG meter 20. However, it should be appreciated that the form of connection element 66 may vary in alternative embodiments as the form of connection element 30 changes. For example, it is contemplated that the engagement between connection elements 30 and 66 may utilize other types of USB connections along with other types of electronic connectors known in the art. As a further alternative, it is contemplated that connection elements 30 and 66 can each define sensor elements that interact with one another when bG meter 20 is positioned in internal receptacle 64. In this embodiment, the interaction between connection elements 30 and 66 can activate an alternative communication interface between bG meter 20 and docking device 50. For example, bG meter 20 and docking device 50 can each include wireless transceivers that are activated upon interaction of the sensors. The wireless transceivers interact with each other to define the communication interface between bG meter 20 and docking device 50. In one form, the sensors are defined by external contacts that engage with each other when bG meter 20 is positioned in internal receptacle 64.

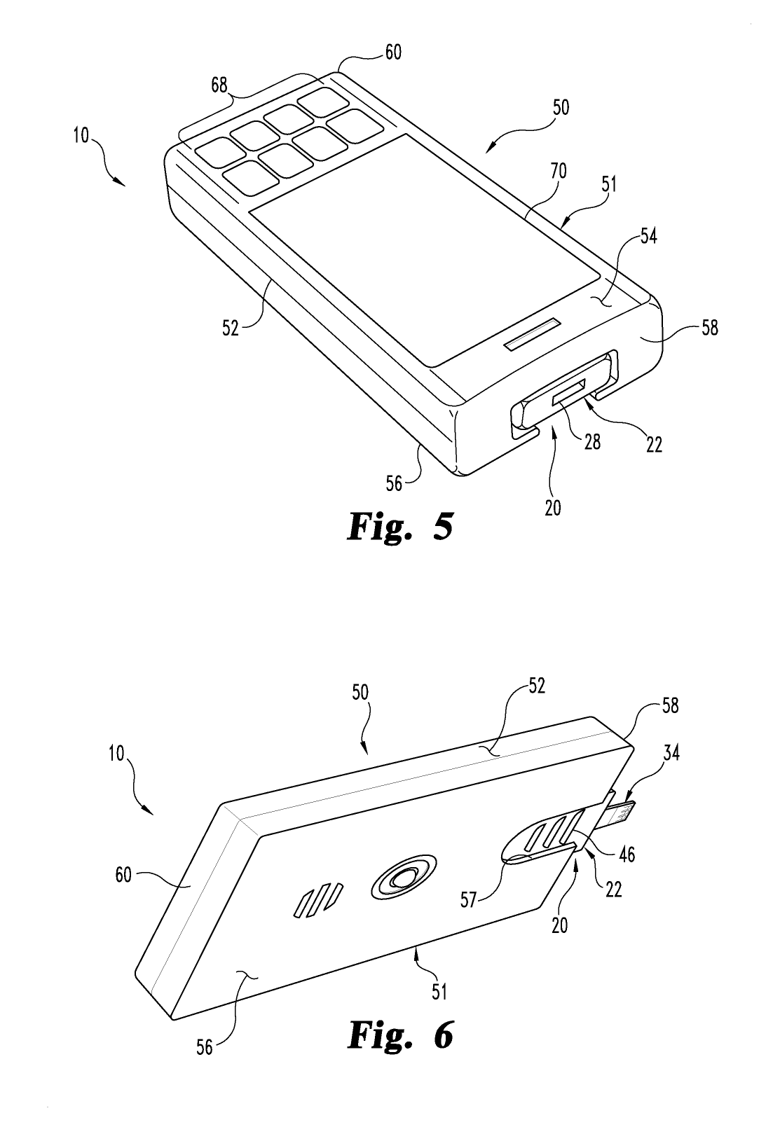

Referring generally to FIGS. 5 and 6, system 10 is illustrated with bG meter 20 positioned in internal receptacle 64 of docking device 50. In the illustrated embodiment, housing 22 of bG meter 20 is substantially surrounded by housing 51. In another non-illustrated embodiment, housing 22 of bG meter 20 is completely surrounded by housing 51. Additionally, docking device 50 substantially retains its external profile and size when bG meter 20 is positioned in internal receptacle 64. More particularly, bG meter 20 is positioned substantially within the external profile of docking device 50. To that end, the external profile, size and shape of docking device 50 are generally not affected by the positioning of bG meter 20 in internal receptacle 64. As further illustrated in FIG. 6, surface 56 of docking device 50 includes an opening 57 extending therethrough into communication with internal receptacle 64 adjacent to receiving portion 62. Opening 57 exposes a portion of bG meter 20 to assist a user in its positioning into and removal from internal receptacle 64. In the illustrated embodiment, meter housing 22 includes a plurality of ridges 46 positioned along a surface of housing 22 that faces opening 57 adjacent to test element port 28. Ridges 46 are generally structured to enhance a user's grip on bG meter 20 when it is being positioned relative to docking device 50. When bG meter 20 is positioned in internal receptacle 64 the ridges 46 are generally positioned adjacent to and accessible through opening 57. In other non-illustrated embodiments, it is contemplated that ridges 46 can be replaced by one or more other grip enhancing features, including for example, knurling, teeth, indentations, serrations or handles, just to name a few possibilities.

Still, in other embodiments it is contemplated that ridges 46 are absent from housing 22 of bG meter 20. In these embodiments, a user can grip housing 22 adjacent to test element port 28 when positioning bG meter 20 in internal receptacle 64. Docking device 50 may include another arrangement for facilitating removal of bG meter 20 from internal receptacle 64. In one non-illustrated example, docking device 50 includes an ejection mechanism engageable with bG meter 20 to facilitate its removal from internal receptacle 64. For example, the ejection mechanism can include a lever arm linked to a depressible ejection button positioned on housing 51 of docking device 50. In this arrangement, depression of the ejection button by a user actuates the lever arm which in turns applies a removal force on bG meter 20. In other non-illustrated embodiments, it is contemplated that docking device 50 includes one or more elements for securing bG meter 20 in internal receptacle 64. Examples of elements for securing bG meter 20 in internal receptacle 64 include latches, straps, clasps and clamps, each of which can be positioned on housing 51 adjacent to receiving portion 62. As a further example, a ball-detent mechanism can be utilized for securing bG meter 20 in internal receptacle 64.

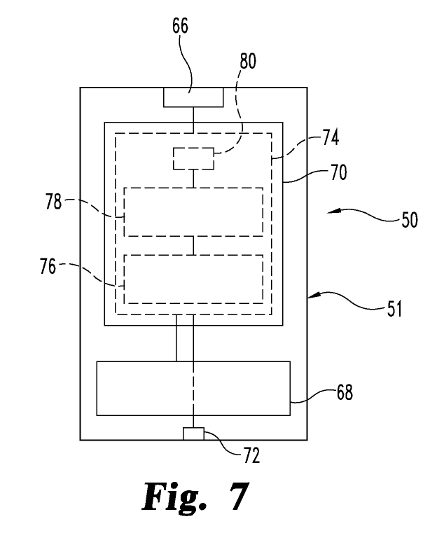

FIG. 7 provides one embodiment of a schematic of docking device 50. Docking device 50 includes a controller 74, memory 76 associated with controller 74, a programmable processor 78 associated with controller 74 and connected with memory 76, and a real-time clock 80 associated with controller 74 and connected with processor 78. Display 70 is connected with processor 78 with, for example, a display driver, and operable to provide a user readable display of output from processor 78. User entry means 68 is connected with processor 78 and accessible by the user to provide input to processor 78. Processor 78 is further programmable to receive input commands from user entry means 68 and provide an output that responds to the input commands.

Processor 78 is connected with connection element 66 and operable to process and record data in memory 76 relating to information stored on memory 40 of bG meter 20. Processor 78 is also operable to produce a representation of the information stored in memory 40 of bG meter 20 on display 70. In an exemplary form, the information stored in memory 40 of bG meter 20 is bG measurement data, although other types of information are contemplated. In one embodiment, one or both of processor 78 of docking device 50 and processor 42 of bG meter 20 is operable to permit an exchange of information between docking device 50 and bG meter 20 in response to bG meter 20 being positioned in internal receptacle 64. In alternative embodiments, the information exchange is generally seamless; that is, it occurs generally automatically without user interaction with entry means 68 or after a single user interaction with entry means 68. It should be appreciated that processor 42 of bG meter 20 is operable to process and record data in memory 40 relating to information which is provided from docking device 50. In one exemplary form, the information provided from docking device 50 is related to the calendar stored in memory 40 of bG meter 20, although other types of information are contemplated. Processor 78 is also operable to interface with one or more of controller 38 and processor 42 of bG meter 20 to facilitate use of bG meter 20 when it is positioned in internal receptacle 64. For example, input commands can be entered with user entry means 68 and passed to processor 42 of bG meter 20. Processor 78 is also operable to produce representations on display 70 of docking device 50 that correspond to representations that are produced on display 24 of bG meter 20. In this regard, bG meter 20 remains fully functional when it is positioned in internal receptacle 64. Accordingly, bG meter 20 can be used to measure bG levels either alone or in combination with docking device 50.

While not previously discussed, docking device 50 may also include a connection element 72 which, in one embodiment, is positioned on end 60 of housing 51 opposite of receiving portion 62. Connection element 72 may be, for example, a mini-USB port or plug, although other variations in the form of connection element 72 are contemplated. Processor 78 is also connected with connection element 72 and operable to control the transfer of information to and from docking device 50 via connection element 72. In one form, connection element 72 may be engaged with a corresponding connection element on a secondary device, such as a PC, to facilitate communication between the secondary device and docking device 50. In this arrangement, it is contemplated that the secondary device could also communicate with bG meter 20 when it is positioned in internal receptacle 64.

Controller 74 may be comprised of one or more components configured as a single unit or of multi-component form. Controller 74 may be programmable, a state logic machine or other type of dedicated hardware, or a hybrid combination of programmable and dedicated hardware. One or more components of controller 74 may be of the electronic variety defining digital circuitry, analog circuitry, or both. As an addition or alternative to electronic circuitry, controller 74 may include one or more mechanical or optical control elements.

In one embodiment including electronic circuitry, controller 74 includes an integrated processor 78 operatively coupled to one or more solid-state memory devices defining, at least in part, memory 76. For this embodiment, memory 76 contains operating logic to be executed by a processor 78 that is a microprocessor and is arranged for reading and writing of data in memory 76 in accordance with one or more routines of a program executed by microprocessor 78.

Memory 76 may include one or more types of solid-state electronic memory and additionally or alternatively may include the magnetic or optical variety. For example, memory 76 may include solid-state electronic Random Access Memory (RAM), Sequentially Accessible Memory (SAM) (such as the First-In, First-Out (FIFO) variety or the Last-In First-Out (LIFO) variety), Programmable Read Only Memory (PROM), Electrically Programmable Read Only Memory (EPROM), or Electrically Erasable Programmable Read Only Memory (EEPROM); or a combination of any of these types. Also, memory 76 may be volatile, nonvolatile or a hybrid combination of volatile and nonvolatile varieties. Some or all of memory 76 can be of a portable type, such as a disk, tape, memory stick, cartridge, code chip or the like. Memory 76 can be at least partially integrated with processor 78 and/or may be in the form of one or more components or units.

In other embodiments, it is contemplated that the docking device 50 may utilize a removable memory key that is pluggable into a socket or other receiving means (not shown) of housing 51, and which communicates with the memory or controller of docking device 50 to provide information relating to calibration codes, measurement methods, measurement techniques, and information management. Examples of such removable memory keys are provided above.

In one embodiment, memory 76 of docking device 50 includes a calendar that stores a schedule of events which may be representative of times for measuring bG levels, taking medications, visiting a physician and performing other daily tasks, including going to work, school and meetings, just to name a few possibilities. In this embodiment, processor 78 is programmed so that an alarm or other indicator on docking device 50 audibly, visually or by vibration alerts the user to perform each event according to the schedule. Once the event has been completed, a user may provide an indication of same with user entry means 68 and processor 78 is programmed to process and record the indication in memory 76. In one form, user entry means 68 is utilized for entering events into the calendar, although it is also contemplated that a secondary device, such as a PC, could be utilized for scheduling events in the calendar. As discussed in further detail below, it is contemplated that docking device 50 can be used for scheduling events in the calendar stored in memory 40 of bG meter 20. In this regard, it should be appreciated that the events stored in memory 76 of docking device 50 may correspond to one or more of the events stored in memory 40 of bG meter 20.

Besides memory 76, controller 74 may also include clock 80, display 70, and entry means 68 associated therewith, along with signal conditioners, filters, limiters, Analog-to-Digital (A/D) converters, Digital-to-Analog (D/A) converters, communication ports, or other types of operators as would occur to those skilled in the art to implement the present invention. In the embodiment illustrated in FIGS. 1 and 5, for example, entry means 68 is defined by a plurality of push-button input devices, although entry means 68 may include one or more other types of input devices like a keyboard, mouse or other pointing device, touch screen, touch pad, roller ball, or a voice recognition input subsystem. Display 70 may include one or more output means like an operator display that can be of a Cathode Ray Tube (CRT) type, Liquid Crystal Display (LCD) type, plasma type, Organic Light Emitting Diode (OLED) type, a printer, or the like. Other input and display means can be included such as loudspeakers, voice generators, voice and speech recognition systems, haptic displays, electronic wired or wireless communication subsystems, and the like.

Docking device 50, either alone or in connection with its interface with bG meter 20, is generally operable to perform a plurality diabetes management functions, which can include any actions related to the schedule, dietary intake, or dietary planning of a diabetic; set-up and configuration of bG meter 20 or docking device 50; analysis of bG measurement data; insulin delivery; general advice or education regarding diabetes management and treatment planning; and/or interaction between a diabetic and third parties, such as healthcare professionals, just to name a few possibilities. More particular examples of diabetes management functions are provided below, although it is contemplated that other functions performed by docking device 50 and described elsewhere herein may also be examples of diabetes management functions. As docking device 50 is configured to perform certain diabetes management functions, it may include features in addition to those described above. In this regard, such additional features are also described below. It should be appreciated that docking device 50 is not precluded from being operable to perform some or all of the functions described herein, or from including some or all of the additional features described below, regardless of such features being described in connection with certain functions.

In one embodiment, docking device 50 may include a communication link for wireless connection with a secondary device, such as a personal computer, modem, local area network, or the worldwide web. Examples of wireless connections can include WiFi, bluetooth, and SMS connections, just to name a few possibilities. In one embodiment, the wireless connection can be used for remote viewing and/or analysis of data, such as bG measurements, stored in memory 76 of docking device 50 or memory 20 of bG meter 20 when bG meter 20 is positioned in internal receptacle 64. The wireless connection can also be utilized for downloading programming updates for one or both of docking device 50 and bG meter 20. When the programming update is for bG meter 20 and it is not interfaced with docking device 50 when the update is received, it is contemplated that docking device 50 can store the update in memory 76 until the next time bG meter 20 is positioned in internal receptacle 64 and interfaces with docking device 50. Once bG meter 20 interfaces with docking device 50, processor 78 is operable to update bG meter 20 with any updates that have been received by the wireless connection and stored in memory 76 since the last time bG meter 20 was interfaced with docking device 50. The wireless connection can also be utilized to exchange messages between the user of docking device 50 and another party, such as a doctor, caseworker, or parent, just to name a few possibilities.

In another embodiment, docking device 50 is operable to configure or set-up one or more aspects of bG meter 20. For example, with bG meter 20 positioned in internal receptacle 64, input can be provided with user entry means 68 to configure or modify various operating aspects of bG meter 20. Such aspects can include, without limitation, the settings of display 24 or programming of processor 42. In another form, docking device 50 provides calibration data to bG meter 20. In this form, processor 42 is operable to process the calibration data and implement modifications or other appropriate actions which are necessary to properly calibrate bG meter 20 to ensure accurate bG measurements. In one form, the calibration data can include information regarding test strips 34 used by bG meter 20. As indicated above, processor 78 of docking device 50 is also operable to provide programming updates to bG meter 20 which are processed and implemented by processor 42.

Processor 78 of docking device 50 is also operable to create, arrange or modify the events that are scheduled in the calendar stored in memory 40 of bG meter 20 and memory 76 of docking device 50. In one form, a scheduler program is provided on docking device 50 that can be used to create a schedule that is uploaded onto bG meter 20 each time bG meter 20 is positioned in internal receptacle 64. Additionally, the schedule created by the scheduler program may also be stored in memory 76 of docking device 50. As a variation however, it is contemplated that the schedule stored in memory 76 of docking device 50 can be different from the schedule stored in memory 40 of bG meter 20. It should be appreciated that the schedule developed by the scheduler program can wholely replace an earlier schedule stored in memory 40, 76, or processor 42, 78 can be operable to make appropriate changes to the earlier schedule according to the later provided schedule.

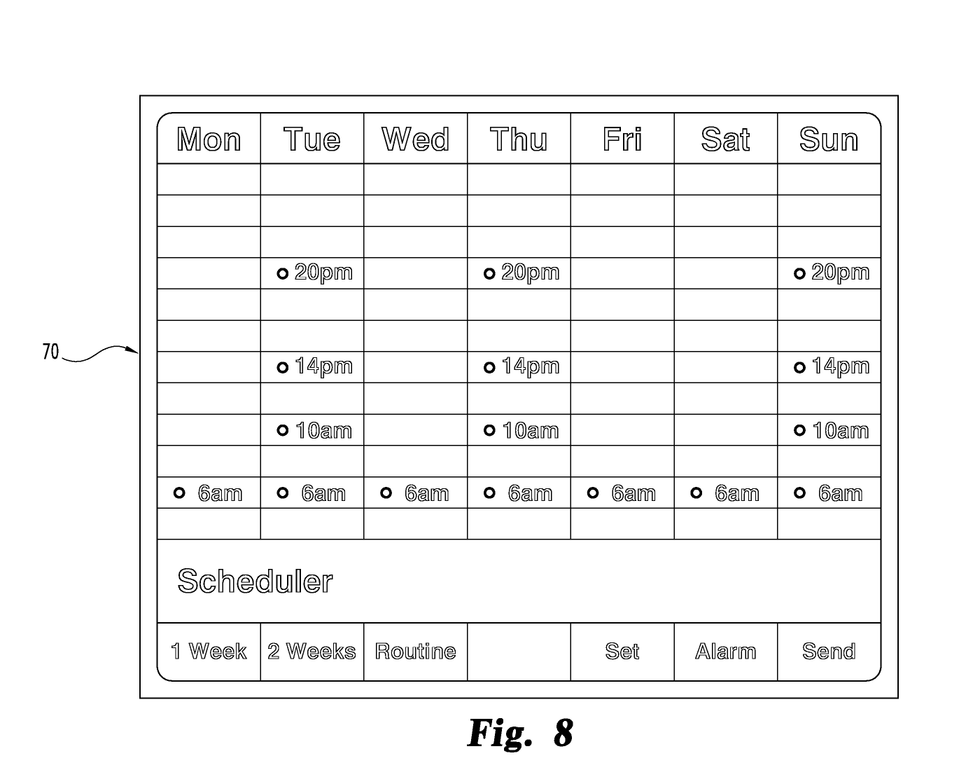

In FIG. 8 there is provided an illustration of display 70 with the scheduler program running. User entry means 68 can be used to set alarms at various times to provide a reminder for performing some event. The events can be representative of appointments, meetings, meals or bG measurements, just to name a few possibilities. It is also contemplated that a unique alarm can be associated with each event. For example, alarms corresponding to bG measurements could be defined by both auditory and vibratory output by bG meter 20 or docking device 50, while alarms corresponding to a meeting could be defined by only one of an auditory or vibratory output. As a further variation, the auditory output can be defined to provide a distinct sound, tune, rhythm or sequence in association with various events. Once a new schedule has been completed, instruction can be provided through user entry means 68 to send the schedule to bG meter 20. If bG meter 20 is not positioned in internal receptacle 64 upon completion of the schedule, the schedule can be stored in memory 76 until the next time bG meter 20 and docking device 50 interface. In the illustrated form, the scheduler program can facilitate programming of a schedule in one or two week increments, although other variations are contemplated. Additionally, user entry means 68 can be used to select a routine schedule which is stored in memory 76 and is representative of a typical day of the user. In this form, the routine schedule can serve as a default schedule which eliminates the need for the user to create an entirely new schedule every time they return to their typical daily routine from some alternative routine, such as a vacation routine.

Other variations in arranging the schedule stored in memory 40 of bG meter 20 are contemplated. For example, in one alternative, when bG meter 20 interfaces with docking device 50, the calendar stored in memory 40 of bG meter 20 can be produced on display 70. In this arrangement, user entry means 68 can be used to navigate through the calendar, store events and specify alarm settings as desired. Once the schedule is completed, it can be saved in memory 40 of bG meter 20. Moreover, it is also contemplated that processor 78 could be operable to carry changes made to the calendar stored in memory 40 of bG meter 20 over to the calendar stored in memory 76 of docking device 50.

Additional interaction between the calendars of docking device 50 and bG meter 20 is also contemplated. For example, in one form, the calendar of docking device 50 can be updated to reflect the user's performance, or lack thereof, of each of the events scheduled. In this form, in response to an alarm, a user can provide an indication with user entry means 26 to bG meter 20 that the event corresponding to the respective alarm was performed. Processor 42 is operable to store the indication in memory 40 and transfer the indication to docking device 50 when bG meter 20 interfaces therewith. Processor 78 of docking device 50 is operable to process the indication and associate it with a corresponding event entered in the calendar stored in memory 76. Processor 78 is further operable to provide on display 70 a modified representation of the calendar stored in memory 76 which indicates which events have been performed. As an example, the representation on display 70 could represent a diabetic's compliance with a bG testing schedule.

As indicated above, docking device 50 is also operable with bG meter 20 to analyze bG measurement data which is stored in memory 40 of bG meter 20. When bG meter 20 is positioned in internal receptacle 64 and interfaced with docking device 50, processor 78 is operable to process and record the bG measurement data stored in memory 40 of bG meter 20. The bG measurement data may be representative of each bG measurement taken by bG meter 20 over the period of time since the bG meter 20 was last interfaced with docking device 50. It is contemplated that the period of time could reflect hourly, daily, weekly, monthly or even longer intervals, just to provide a few examples. Processor 78 is operable to store the bG measurement data in memory 76 of docking device 50. Additionally, processor 78 is operable to provide a representation of the bG measurement data from memory 40 on display 70. In one variation, processor 78 is operable to aggregate bG measurement data from memory 40 along with other bG measurement data stored in memory 78 and provide a representation of the aggregation on display 70. The aggregation of bG measurement data can include all bG measurements taken within a period of time, such as a day, week or month. Additionally, display 70 could provide a representation of the aggregation of bG measurement data over each of these periods and user entry means 68 can be utilized to navigate and view each representation.

Graphical and textual representations of the bG measurement data on display 70 are contemplated. As used herein, a graphical representation may include one or more textual representations in combination with one or more graphical representations of or related to the data. The textual presentation may be included with the graphical presentation, or in one or more alternating displays of the graphical representation. In one embodiment, a graphical representation of bG measurement data is produced by processor 78 on display 70 so that the user is provided with a visual representation of the bG measurement data. If display of the bG measurements is desired, then the display thereof on display 70 may be produced immediately upon a single user interaction with entry means 68 indicating that the display is desired, or automatically after a pre-determined delay following the interface of bG meter 20 with docking device 50. Alternatively, the bG measurement data processed by processor 78 of docking device 50 can be accessed by a user through one or more menus provided on display 70 and navigated by user entry means 68.

It is contemplated that the graphical and textual representations of the bG measurement data produced on display 70 by processor 78 include any one or combination of xy-graphs, bar graphs, data plots, pie charts, or other suitable graphical representation to represent the bG measurements. In addition, the graphical or textual representations can be color coded to facilitate user interpretation of the results. For example, if one or more of the measured bG values is outside a recommended pre-defined range of blood glucose limits, the display can be presented with a warning indicator or in a warning color, such as red. If the one or more of the measured bG values is near or slightly above or below the pre-defined range of blood glucose limits, display 70 can be presented with a cautionary indicator or in a cautionary color, such as yellow. If all the associated bG measurements are within the recommended range, then display 70 can be presented with a satisfactory indicator or color, such as green. Further details regarding display and representations of bG measurement data are described in U.S. patent application Ser. No. 12/481,965, filed 10 Jun. 2008, the disclosure of which is incorporated herein by reference in its entirety.

In one embodiment, processor 78 is operable to perform additional functions in response to processing the bG measurement data stored on bG meter 20 or the bG measurement data stored on bG meter 20 in aggregate with other bG measurement data stored in memory 76 of docking device 50. For example, in one form docking device 50 includes an insulin bolus calculator and processor 78 is operable to determine a suitable insulin dosage with the insulin bolus calculator based on the bG measurement data. Dietary information which the user can enter and store in memory 76, further details of which are provided below, may also be considered when determining the insulin dosage. In this arrangement, docking device 50 may also provide bolusing instructions which correspond to the insulin dosage determined by the bolus calculator to one or more secondary devices, such as insulin delivery devices. Examples of insulin delivery devices include, without limitation, insulin pumps, pens and syringes. In one embodiment, docking device 50 can automatically provide the bolusing instructions to the secondary device(s) in response to determining the insulin dosage.

In another embodiment, processor 78 is operable to create a prompt on display 70 for the user to confirm that the bolusing instructions should be sent. The user can provide an input with user entry means 68 which confirms or cancels the bolusing instructions. In an alternative embodiment, the insulin dosage is not automatically determined by docking device 50. Instead, the user can use user entry means 68 to open and control the insulin dosage calculator and independently determine the appropriate insulin dosage. In a manner similar to that described above, the user can instruct docking device 50 to provide bolusing instructions to the secondary device that correspond to the appropriate insulin dosage determined by the bolus calculator. While not previously mentioned, it should be appreciated that docking device 50 can provide the bolusing instructions to the secondary device(s) through a wired or wireless connection.

Docking device 50 can interface with the secondary devices to determine and/or configure various operating aspects of these devices as well. For example, in one form, processor 78 is operable to regulate the rate at which the secondary device, such as an insulin pump, delivers the insulin dosage to a user of the pump. Processor 78 can also determine the date of expiration of the pump or the amount of insulin remaining in the pump and/or calculate the estimated amount of time left before the pump distributes the remaining insulin. It is also contemplated that processor 78 can provide an audible or visual indication of these aspects of the pump or some other secondary device to the user. Still, it is contemplated that processor 78 can perform other management and configuration functions relative to the secondary devices beyond those specifically set forth herein.

It is also contemplated that docking device 50 can store insulin delivery data, such as doses or dosage amounts. In one form, processor 78 is operable to store the insulin delivery data in memory 76 of docking device 50 according to the time and date when the insulin was delivered. When docking device 50 provides bolusing instructions to the secondary device(s) as described above, it is contemplated that the insulin delivery data is automatically stored in memory 76 when the bolusing instructions are provided to the secondary device(s). Alternatively, a user can independently provide input through user entry means 68 which is indicative of the insulin delivery data, regardless of whether docking device 50 provides bolusing instructions to the secondary device(s). Processor 78 may be further operable to provide textual and graphical representations of the insulin delivery data, which may include any one or combination of xy-graphs, bar graphs, data plots, pie charts, or other suitable graphical representation to represent the insulin delivery data. In one form, the textual and graphical representations could display insulin doses delivered over a daily, weekly or monthly interval, just to name a few possibilities. In yet a further embodiment, processor 78 is operable to compare the insulin delivery data with other information stored in memory 76, such as the bG measurement data, in a single graphical or textual representation.

In addition to the foregoing, it is contemplated that processor 78 could provide user advice in response to processing the bG measurement data. The advice could be in the form of an audible, visual or audio-visual presentation, just to name a few possibilities. The advice can indicate whether the processed bG measurements are outside a recommended range, within a recommended range, or moderately outside or approaching limits of a recommended range. The advice can also present a recommended action or course of actions to the user based on the bG measurement data. For example, if one or more of the bG measurements is severely outside the recommended range, the advice can recommend dietary changes and changes to insulin dosage per a doctor's advice. If one or more of the bG measurements is moderately outside the recommended range, then the advice can recommend dietary changes to assist the user in obtaining future bG measurements within the recommended range. The advice can also provide positive feedback to the user if the bG measurements are within the recommended range and no dietary or insulin dosage changes are needed.

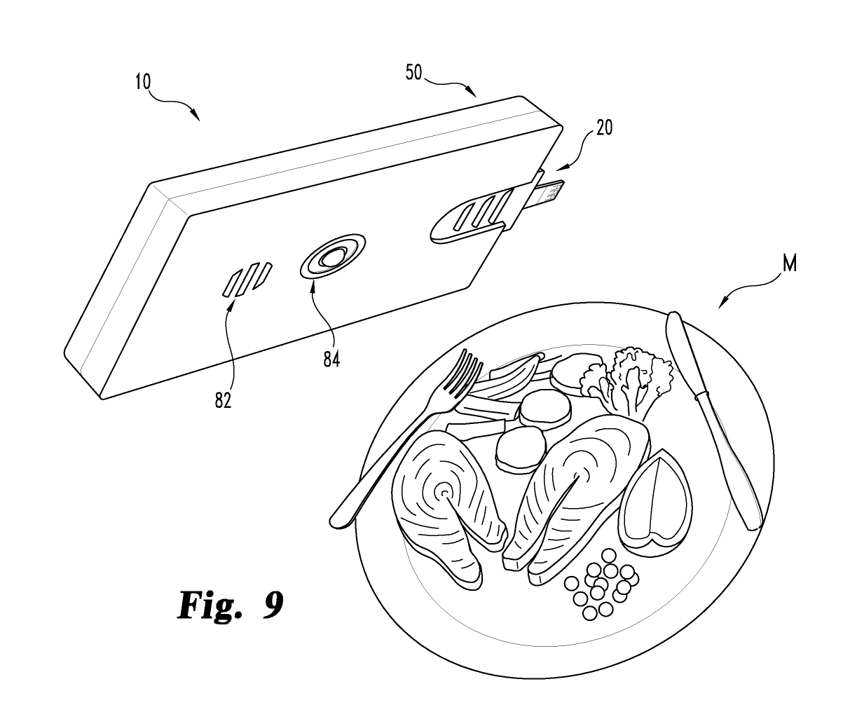

Docking device 50 can also be operable to record, store, manage and organize information regarding the user's dietary and daily routines and other aspects of the user's health. As illustrated in FIG. 9 for example, docking device 50 includes an audio recorder 82 and an image capture device 84. Image capture device 84 can be a digital camera or a video camera. Processor 78 is connected to recorder 82 and device 84 and is operable to process input from recorder 82 and device 84 and record it in memory 76. In FIG. 9, docking device 50 is positioned adjacent to a meal M where device 84 can capture an image of meal M and a user can record narrative details of the meal M with recorder 82. It should be appreciated however that recorder 82 and device 84 can be used for recording information that is not related to meals of the user. For example, details regarding exercise activities performed by the user could be captured by recorder 82 and/or device 84. In addition to recorder 82 and device 84, it is contemplated that the user can enter additional dietary and daily activity details as well as general health related details into memory 76 with user entry means 68. For example, nutritional information of the food which the user consumes could be entered with user entry means 68 and stored in memory 76. Examples of nutritional information that could be entered with user entry means 68 can include numerical values for caloric and carbohydrate intake. In one form, it is contemplated that the carbohydrate value could be further broken down into values for sugar and fiber intake. To assist with entering the nutritional information, docking device 50 can be provided with a food database which is navigable with entry user means 68 to determine the nutritional information of specific foods consumed by the user. It is also contemplated that details regarding activities performed by the user could be recorded. For example, if the activity was some sort of exercise, details regarding the length and difficulty of the exercise could be entered. Still, further examples of information that could be recorded and stored in memory 76 include the user's weight and non-insulin medications. In one or more forms, processor 78 is operable to record and associate relevant time and date details with the information that is stored in memory 76 regarding the user's dietary and daily routines and other aspects of the user's health.

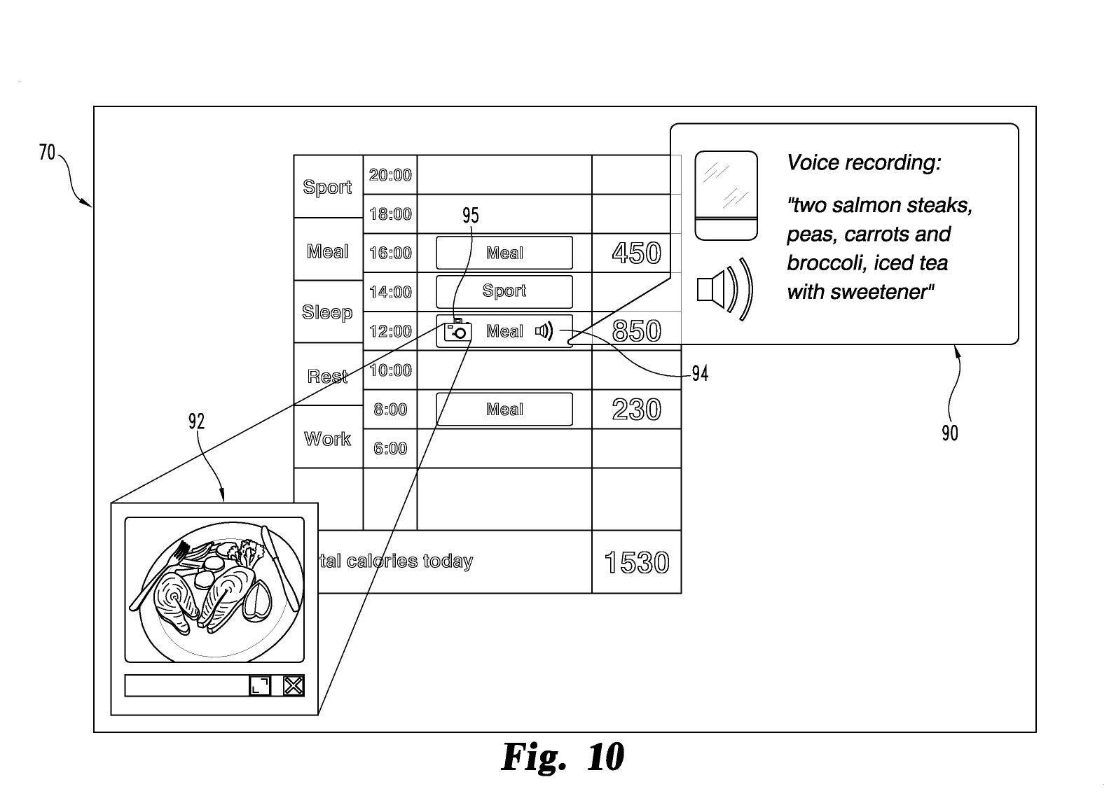

In a further embodiment, processor 78 is operable to access the information that is stored in memory 76 regarding the user's dietary and daily routines and other aspects of the user's health and generate an interactive diary log of such information. Processor 78 is also operable to display the diary log on display 70. It is also contemplated that insulin dosage information and bG measurement data can be incorporated into the diary log generated by processor 78. One example of a diary log displayed on display 70 is illustrated in FIG. 10. The diary log provides details, including the time, of performance of certain recorded events during the period which is displayed, which happens to be one day. However, it should be appreciated that the diary log could be configured to provide information for shorter or longer periods of time. Entries for three meals and one sport activity are illustrated in FIG. 10. Additionally, the calories consumed at each meal as well as the total number of calories consumed during the displayed day are provided. The diary log also includes a number of icons which can be selected by the user with user entry means 68 to reveal additional information. For example, when a user selects the volume icon 94, a voice recording is played. In the illustrated embodiment, the voice recording is displayed in dialogue box 90 and corresponds to the narrative description of meal M that was recorded by the user with recorder 82. As another example, when a user selects the camera icon 95, an image or a video is provided. In the illustrated embodiment, the image is provided and displayed in graphic box 92 and corresponds to the image of meal M that was captured by the user with device 84. In yet another example, a user can select the “Meal” and “Sport” icons to review additional information which has been entered regarding these events, even if such information was not recorded with recorder 82 or device 84. It should be appreciated that that the diary log can provide additional information beyond that illustrated in FIG. 10. For example, in one form, an indication of the total amount of carbohydrates consumed within a period of time can be provided. As another example, the collective amount of time the user spent performing certain activities, such as exercise, can be provided.

Processor 78 may be further operable to provide textual and graphical representations of the nutritional information stored in memory 76. For example, the representations could depict the amount of carbohydrates or calories that the user consumes within a period of time, such as a day, week or month, just to provide a few examples. The representations may include any one or combination of xy-graphs, bar graphs, data plots, pie charts, or other suitable graphical representation to represent the nutritional information. It is further contemplated that the representation could display the nutritional information in combination with one or both of the bG measurement data and the insulin dosage data recorded in memory 76.

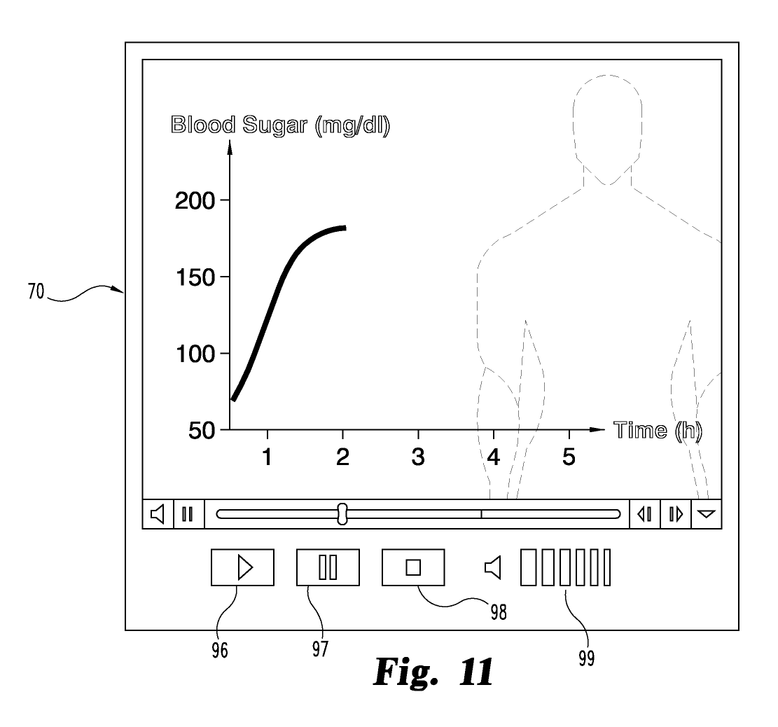

It is also contemplated that docking device 50 could be provided with educational or training programs designed to assist the user in developing and following a diabetes management program. Among other things, the programs can provide basic educational information on diabetes, bG measuring and analysis, and the interplay between dietary habits and intake and proper diabetes management. In one form, the training programs can be provided in the form of a video. For example, as illustrated in FIG. 11, a media player showing a video on bG levels is provided on display 70. User entry means 68 can be utilized to control the video played by the media. For example, in response to input by user entry means 68, processor 78 is operable to activate one of the play 96, pause 97, stop 98 and volume 99 controls which operate the media player such as shown in FIG. 11.

It is further contemplated that docking device 50 could be provided with audible assistance with data management, such as is described in one or more of the following U.S. Patent Application Publications, the disclosures of which are incorporated herein by reference in their entireties: US 2006/0277048; US 2008/0172235; and US 2008/0243758.

As indicated above, it is contemplated that bG meter 20 can be coupled with a PC. Additionally, docking device 50, either alone or with bG meter 20 positioned in internal receptacle 64, can also be coupled with a PC through a wired or wireless connection. The PC includes software which interacts with bG meter 20 and docking device 50 and is operable to perform one or more of the diabetes management functions that docking device 50 performs. For example, the software can process and record bG measurement data from bG meter 20, perform an analysis of the bG measurement data and provide an output, such as a graphical representation, of the bG measurement data. It is also contemplated that the PC software is operable to organize and modify the events scheduled in the calendar stored in memory 40 of bG meter 20 or memory 76 of docking device 50. Still, in further forms it is contemplated that the PC software is operable to configure one or more of the operating parameters of bG meter 20 or docking device 50. As an example, the PC software can provide updates to one or both of bG meter 20 and docking device 50 which are first downloaded onto the PC from the internet or some other electronic media, such as a CD or DVD. In addition to the foregoing, it is contemplated that the PC software can be operable to download information from docking device 50 and transfer it to appropriate health management related databases. For example, in one form it is contemplated that certain information stored on docking device 50 can be transferred to a relevant insurance database. In another form, it is contemplated that relevant information stored on docking device 50 can be downloaded into an electronic health record of the patient.

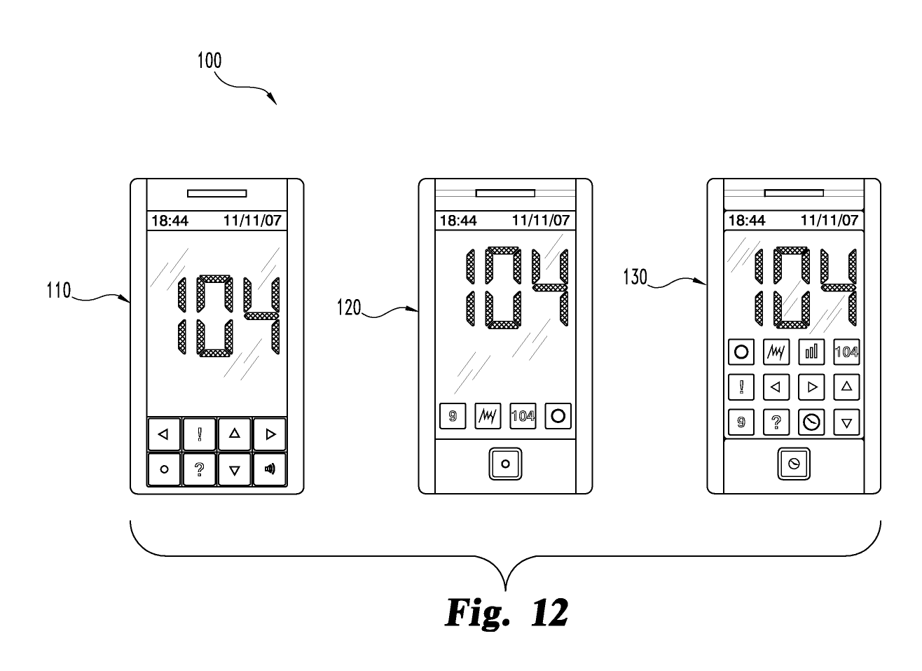

In a further embodiment of the present application illustrated in FIG. 12, a system 100 includes a plurality of docking devices 110, 120, 130, although additional docking devices are contemplated. Docking devices 110, 120 and 130 each include an internal receptacle (not shown) that is structured to receive bG meter 20 as described above with respect to docking device 50. Additionally, docking devices 110, 120 and 130 can also each interface with bG meter 20 as described above with respect to bG meter 20 and docking device 50. Docking devices 110, 120 and 130 can include one or more of the features described above with respect to docking device 50, but are each independently configured to perform a unique set of diabetes management functions relative to each other. As used herein, the term “unique” means that each of the docking devices 110, 120 and 130 performs diabetes management functions in addition to or in lieu of those performed by another docking device.

In one form, each of the docking devices 110, 120 and 130 is configured to correspond to the specific needs of a user likely to use the respective docking device 110, 120 and 130. For example, in one form, one of the docking devices 110, 120 and 130 is configured to be used by an insulin dependent diabetic, a second of the docking devices 110, 120 and 130 is configured to be used by a physician, and a third of the docking devices 110, 120 and 130 is configured to be used by a dietician. Several exemplary differences between the functionality of the docking devices in this arrangement can be present. For example, the first one of the one of the docking devices 110, 120 and 130 can include a calendar, which provides reminders for checking bG levels or taking insulin, which is not included by the second and third docking devices. As another example, the second one of devices 110, 120 and 130 can be configured to provide sophisticated graphical analysis of bG measurement data useful to the physician, but not otherwise useful to the insulin-dependent diabetic or the dietician. To that end, the other two docking devices are not configured to provide this type of bG measurement data analysis. Still, further examples of differences between the docking devices 110, 120 and 130 are contemplated by the subject application. Additionally, it should be appreciated that devices 110, 120 and 130 can be configured for users beyond those described above.

While not specifically described above, it should be appreciated that bG meter 20 can be used to measure blood glucose values as a stand-alone device and/or when it is positioned in receptacle 64 of docking device 50. For example, when used alone, a user can control bG meter 20 with entry means 26, if necessary, and the information related to the functions performed by bG meter 20, including the results of blood glucose measurements, are provided on display 24. In the arrangement where bG meter 20 is positioned in receptacle 64 of docking device 50 however, a user can control bG meter 20 with entry means 68, if necessary, and the information related to the functions performed by bG meter 20, including the results of blood glucose measurements, are provided on display 70 of docking device 50. In one form of this arrangement, it is contemplated that the information on display 70 of docking device 50 is the same as or substantially similar to the information provided on display 24 of bG meter 20 when bG meter 20 is used alone. Moreover, the information may be provided on display 70 in a format that is the same as or substantially similar to the format in which the information is provided on display 24 of bG meter 20. Additionally, the manner for operating and controlling bG meter 20 with docking device 50 may be the same as or substantially similar to the manner in which bG meter 20 is operated and controlled when bG meter 20 is used alone. As an additional or alternative feature of the arrangement where bG meter is positioned in receptacle 64 of docking device 50, it is contemplated that the ability for a user to control bG meter 20 with entry means 68 of docking device 50 may generally occur automatically, or seamlessly, as a result of the communication interface between bG meter 20 and docking device 50. Similarly, the information related to the functions performed by bG meter 20, including the results of blood glucose measurements, may generally be provided automatically, or seamlessly, on display 70 of docking device 50 as a result of the communication interface between bG meter 20 and docking device 50. Further details regarding various configurations for displaying blood glucose measurements on one or both of bG meter 20 and docking device 50 are described in U.S. patent application Ser. No. ______, titled “Blood Glucose Management and Interface Systems and Methods” (Attorney Docket No. ROCD-12/WP24855US) and filed on the same date as the subject application, the disclosure of which is incorporated herein by reference in its entirety. However, it also contemplated that entry means 68 and display 70 of docking device 50 will not control bG meter 20 or provide information related to its functions unless first prompted by a user with, for example, entry means 68 of docking device 50.

While the invention has been illustrated and described in detail in the drawings and foregoing description, the same is to be considered as illustrative and not restrictive in character, it being understood that only certain embodiments have been shown and described and that all changes and modifications that come within the spirit of the inventions are desired to be protected. It should be understood that while the use of words such as preferable, preferably, preferred or more preferred utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

1. A system, comprising:
a portable, stand-alone blood glucose meter including a display and a first connection element;
a portable, handheld docking device including a display operable to provide information related to functions performed by said blood glucose meter and a housing defining an external profile of said docking device and an internal receptacle, said internal receptacle including a second connection element engageable with said first connection element; and
wherein said blood glucose meter is positionable in said internal receptacle and a communication interface is defined between said blood glucose meter and said docking device when said blood glucose meter is positioned in said internal receptacle and said first connection element is engaged with said second connection element.

2. The system of claim 1, wherein said blood glucose meter includes a meter housing which substantially corresponds in size and shape to said internal receptacle, said meter housing being substantially surrounded by said internal receptacle and positioned within said external profile of said docking device when said blood glucose meter is positioned in said internal receptacle.

3. The system of claim 1, wherein said housing of said docking device includes a sidewall extending around said housing and between a first surface and a second surface positioned opposite of said first surface, and wherein said internal receptacle is positioned between said first and second surfaces.

4. The system of claim 3, wherein said internal receptacle includes a receiving portion extending through said sidewall of said housing.

5. The system of claim 4, wherein said second surface includes an opening in communication with a portion of said internal receptacle adjacent to said receiving portion.

6. The system of claim 4, wherein said second connection element is positioned in said internal receptacle opposite of said receiving portion.

7. The system of claim 1, wherein one of said first connection element and said second connection element defines a plug and the other of said first connection element and said second connection element defines a port structured to receive said plug.

8. The system of claim 1, wherein said blood glucose meter includes a meter housing defining a test element port for receiving a test element.

9. The system of claim 8, wherein said test element comprises a test strip.

10. The system of claim 8, wherein said first connection element is positioned on said meter housing opposite of said test element port.

11. The system of claim 8, wherein said blood glucose meter includes a memory configured to store blood glucose measurement data.

12. The system of claim 11, wherein said blood glucose measurement data is transferred from said blood glucose meter to said docking device when said blood glucose meter is positioned in said internal receptacle and said first connection element is engaged with said second connection element.

13. The system of claim 12, wherein said blood glucose measurement data is seamlessly transferred from said blood glucose meter to said docking device in response to said blood glucose meter being positioned in said internal receptacle and said first connection element being engaged with said second connection element.

14. The system of claim 12, wherein said docking device is configured to process said blood glucose measurement data and provide an output on said display of said docking device to a user of said docking device after said blood glucose measurement data is processed.

15. The system of claim 14, wherein said output is a graphical representation of said blood glucose measurement data.

16. The system of claim 1, wherein a blood glucose measurement is provided on said display of said blood glucose meter in response to measuring a glucose level with said blood glucose meter when said glucose meter is not positioned in said internal receptacle of said docking device.

17. The system of claim 1, wherein a blood glucose measurement is provided on said display of said docking device in response to measuring a glucose level with said blood glucose meter when said blood glucose meter is positioned in said internal receptacle of said docking device.

18. A system, comprising:
a portable, stand alone blood glucose meter;
a plurality of portable, handheld docking devices each including a housing defining an internal receptacle sized and structured to receive said blood glucose meter and including an interface for communicating with said blood glucose meter when said blood glucose meter is positioned in said internal receptacle; and
wherein each one of said plurality of docking devices is operable to communicate with said blood glucose meter to perform a unique set of diabetes management functions relative to the other of said plurality of docking devices.

19. The system of claim 18, wherein said blood glucose meter and each one of said plurality of docking devices further includes:
a display;
a user entry means for receiving user input;
a memory; and
a programmable processor operatively connected to said display, said user entry means and said memory.

20. The system of claim 19, wherein said memory of said blood glucose meter stores a program for operating said processor of said blood glucose meter, said processor of said blood glucose meter being operable with said program to produce at least one blood glucose measurement, provide said at least one blood glucose measurement on said display of said blood glucose meter and store said at least one blood glucose measurement in said memory of said blood glucose meter.

21. The system of claim 20, wherein said memory of said blood glucose meter stores a schedule of events and said processor of said blood glucose meter is operable with said program to activate a reminder of each of said events to a user.

22. The system of claim 21, wherein at least one of said plurality of docking devices is operable to organize said schedule of events when said blood glucose meter is positioned in said internal receptacle of said at least one docking device.

23. The system of claim 21, wherein said schedule of events includes a plurality of predetermined times for measuring blood glucose levels.

24. The system of claim 19, wherein said user entry means of said blood glucose meter includes a plurality of buttons, and wherein upon activation of a first one of said plurality of buttons by a user said processor of said blood glucose meter is operable with said program to activate a reminder for measuring blood glucose levels after one of a plurality of predefined periods of time.

25. The system of claim 24, wherein said one of a plurality of predefined periods of time is determined by a number of times a user activates said first one of said plurality of buttons.

26. The system of claim 20, wherein said memory of each of said docking devices stores a unique management program for operating said processor of said respective docking device.

27. The system of claim 26, wherein said processor of at least one of said plurality of docking devices is operable to process said at least one blood glucose measurement stored in said memory of said blood glucose meter.

28. The system of claim 27, wherein said processor of said at least one of said plurality of docking devices is further operable to perform one or more of said disease management functions after said at least one blood glucose measurement is processed.

29. The system of claim 28, wherein said disease management functions include at least one of providing a graphical representation of said at least one blood glucose measurement and providing instructions to an insulin delivery device.

30. The system of claim 28, wherein said processor of said at least one of said plurality of docking devices provides a graphical representation of said at least one blood glucose measurement when said blood glucose meter is positioned in said internal receptacle of said at least one of said plurality of docking devices.

31. The system of claim 30, wherein said processor of said at least one of said plurality of docking devices seamlessly provides said graphical representation of said at least one blood glucose measurement in response to said blood glucose meter being positioned in said internal receptacle of said at least one of said plurality of docking devices

32. The system of claim 26, wherein said processor of at least one of said plurality of docking devices is operable to configure said program for operating said processor of said blood glucose meter.

33. The system of claim 19, wherein said user entry means of at least one of said plurality of docking devices is defined by a touch screen, a camera and a voice recorder.

34. A method, comprising:
providing a portable, handheld docking device including a display and a housing defining an external profile of said docking device and an internal receptacle structured to house a stand-alone blood glucose meter;
positioning said blood glucose meter in said internal receptacle while substantially retaining said external profile of said docking device; and
in response to the positioning, transferring information from at least one of said docking device and said blood glucose meter to the other of said docking device and said blood glucose meter.

35. The method of claim 34, wherein said positioning includes engaging a first connection element of said blood glucose meter with a second connection element positioned in said internal receptacle.

36. The method of claim 34, wherein the transferring includes seamlessly receiving blood glucose measurement data with said docking device from said blood glucose meter.

37. The method of claim 36, further comprising, in response to receiving said blood glucose measurement data, processing said blood glucose measurement data with said docking device.

38. The method of claim 37, further comprising, in response to processing said blood glucose measurement data, at least one of producing a graphical representation of said blood glucose measurement data on said display of said docking device and providing instructions with said docking device to an insulin delivery device.

39. The method of claim 34, wherein the transferring includes loading one or more events with said docking device onto a schedule stored in said blood glucose meter.

40. The method of claim 34, wherein providing said docking device includes selecting said docking device from a plurality of portable, handheld docking devices, each of said plurality of docking devices including a display and a housing defining an external profile of said respective docking device and an internal receptacle structured to house said blood glucose meter.

41. The method of claim 40, wherein each of said plurality of docking devices is independently configured to provide a unique set of diabetes management functions relative to the other of said plurality of docking devices.

42. The method of claim 34, which further includes providing a blood glucose measurement on said display of said docking device in response to measuring a blood glucose level with said blood glucose meter when said blood glucose meter is positioned in said internal receptacle of said docking device.

43. The method of claim 34, which further includes:
removing said blood glucose meter from said internal receptacle;
measuring a blood glucose level with said blood glucose meter; and
in response to measuring said blood glucose level, providing a blood glucose measurement on a display of said blood glucose meter.
